(19) United States
(12) Patent Application Publication
(10) Pub. No.: US 2015/0209391 A1
(43) Pub. Date: Jul. 30, 2015

(54) Cancer Treatment Composition

(71) Applicant: Samir Witta, Golden, CO (US)

(72) Inventor: Samir Witta, Golden, CO (US)

(21) Appl. No.: 14/605,126

(22) Filed: Jan. 26, 2015

Related U.S. Application Data
(60) Provisional application No. 61/931,154, filed on Jan. 24, 2014.

(51) Int. Cl. A61K 35/644; A61K 45/06; A61K 31/352; A61K 31/05; A61K 31/12

(52) U.S. Cl. CPC A61K 35/644; A61K 31/05; A61K 31/12; A61K 31/352; A61K 45/06

(57) ABSTRACT

A cancer treatment composition that includes natural substances. The cancer treatment composition functions by inhibiting growth and formation of cancer cells by inhibiting specific pathways. Specifically, the composition provides a combination of Wnt signaling pathway inhibitors and histone deacetylase inhibitors. Preferably, the composition includes therapeutically effective amounts of dimethoxycurcumin and/or quercitin for inhibiting the Wnt signaling pathway, and honokiol and royal jelly for inhibiting histone deacetylase. The composition may further include one or more antitumorigenic compounds such as graviola guanabana, lyceum barbarum, gogi, red reishi, and salvestrol, among others. Thus, the present invention provides a cancer treatment composition having natural substances and compounds.

### CROSS REFERENCE TO RELATED APPLICATION

This application claims the benefit of U.S. Provisional Application No. 61/931,154 filed on Jan. 24, 2014. The above identified patent application is herein incorporated by reference in its entirety to provide continuity of disclosure.

### BACKGROUND OF THE INVENTION

1. Field of the Invention

The present invention relates to compositions for inhibiting the growth and formation of cancer cells. More specifically, the present invention provides a cancer treatment composition comprising natural substances, wherein the composition comprises a combination of Wnt signaling pathway inhibitors and histone deacetylase inhibitors, so as to inhibit the growth and formation of cancer cells.

The Wnt signaling pathways are signal transduction pathways composed of proteins that pass signals through cell surface receptors into a cell. The Wnt pathway leads to regulation of gene transcription. Malfunctions in the Wnt signaling pathways can result in a variety of diseases, such as various types of cancer. Thus, the Wnt signaling pathway is a significant pathway in cancer development and in the maintenance of cancer stem cells. Substances or compounds that interfere with or inhibit the Wnt signaling pathway may be helpful in preventing or limiting the growth and formation of cancer cells via mutations in this pathway.

Similarly, histone deacetylase inhibitors serve to impact the expression of genes leading to favorable signature in cancerous cells or in cells that are at risk to become cancerous. Histone deacetylase inhibitors also affect changes in cells that may help to improve the effect of other modulators of pathways critical in cancer. Some histone deacetylase inhibitors have been shown to impact the cancerous potential of cancer stem cell.

Thus, the Wnt pathway and histone deacetylases are both important for the survival of cancer stem cells. Inhibition of both the Wnt pathway and histone deacetylases serves to halt the growth or survival of cancer stem cells. Accordingly, a combination of both Wnt inhibitors and histone deacetylase inhibitors provides a synergistic effect in limiting the survival and growth of cancer stem cells.

Some patents and publications in the prior art disclose compositions for inhibiting the Hedgehog pathway, such as U.S. Published Patent Application Number 2008/0019961 which provides a composition for treating tumorigenic cells with hedgehog signaling pathway antagonists, and U.S. Patent Number 2013/0224311 which provides methods and composition for treating cancer and for modulating the activity of the Hedgehog pathway. Other patents and publications disclose various compositions and methods for treating cancer, such as U.S. Pat. No. 8,058,258, U.S. Published Patent Application Number 2011/0171219, U.S. Pat. No. 8,318,677, and U.S. Published Patent Application Number 2008/0267947.

These prior art devices have several known drawbacks. Some prior art compositions are directed only towards inhibiting growth and formation of cancer cells from a single pathway. However, cancer cells can grow and proliferate from any of various pathways. These prior art compositions fail to disclose a composition for treating cancer wherein the composition comprises a combination of histone deacetylase inhibitors and Wnt signaling pathway inhibitors. Further, the prior art fails to disclose a composition for cancer treatment that includes natural substances and compounds.

In light of the devices disclosed in the prior art, it is submitted that the present invention substantially diverges in design elements from the prior art and consequently it is clear that there is a need in the art for an improvement to existing cancer treatment compositions. In this regard the instant invention substantially fulfills these needs.

### SUMMARY OF THE INVENTION

In view of the foregoing disadvantages inherent in the known types of cancer treatment compositions now present in the prior art, the present invention provides a new cancer treatment composition wherein the same can be utilized for providing convenience for the user when inhibiting the growth and formation of cancer cells using natural substances and compounds.

It is therefore an object of the present invention to provide a new and improved cancer treatment composition that has all of the advantages of the prior art and none of the disadvantages.

It is another object of the present invention to provide a cancer treatment composition comprising a combination of Wnt signaling pathway inhibitors and histone deacetylase inhibitors.

Another object of the present invention is to provide a cancer treatment composition that utilizes natural substances to inhibit the growth and formation of cancer cells.

Yet another object of the present invention is to provide a cancer treatment composition that includes one or more natural substances having antitumorigenic properties.

Other objects, features and advantages of the present invention will become apparent from the following detailed description taken in conjunction with the accompanying drawings.

### DETAILED DESCRIPTION OF THE INVENTION

The present invention provides a composition for treating cancer. The composition is directed at inhibiting the growth and formation of cancer cells from certain pathways that are significant in the survival of cancer stem cells. Specifically, the composition comprises a combination of Wnt pathway inhibitors and histone deacetylase inhibitors. Thus, the composition is adapted to inhibit the growth and formation of cancer cells that result from certain processes that play significant roles in carcinogenesis. Some embodiments of the present invention further include one or more substances known or thought to have antitumorigenic or anti-cancer properties.

The composition includes one or more Wnt signaling pathway inhibitors for preventing or inhibiting the growth and formation of cancer cells via the Wnt signaling pathway. Any of various Wnt pathway inhibitors can be used. Flavonoids, such as demethoxycurcumin and quercetin have been shown in some studies to have an effect in targeting the Wnt signaling pathway and can serve as Wnt pathway inhibitors. Accordingly, some embodiments of the present invention include therapeutically effective amounts of demethoxycurcumin and/or quercetin for inhibiting the Wnt signaling pathway.

In alternate embodiments of the present invention, the Wnt pathway inhibitor may include salvia cavaleriei. Salvia cavaleriei can be included in the composition in addition to or in place of demethoxycurcumin and/or quercetin for inhibiting the Wnt signaling pathway. Salvia cavaleriei is thought to have anti-tumor activity in a variety of cancer cells. Further, hydnocarpin isolated from Lonicera japonica can be used as a Wnt pathway inhibitor in addition to or in place of demethoxycurcumin, quercetin, and salvia cavaleriei.

In addition to Wnt pathway inhibitors, the composition includes one or more histone deacetylase inhibitors. The composition of the present invention may include any of various histone deacetylase inhibitors. In some embodiments of the present invention, the composition includes honokiol as a histone deacetylase inhibitor. Honokiol is a lignin that can be obtained from the bark and leaves of trees of the genus Magnolia. Honokiol is shown to have use in causing cell death in various forms of cancer such as melanoma, sarcoma, myeloma, and leukemia, among others.

Another histone deacetylase inhibitor is royal jelly. The composition may include royal jelly as the histone deacetylase inhibitor, or may use both honokiol and royal jelly. Royal jelly is a honey bee secretion that is often used as a general health tonic thought to be useful in boosting the immune system. Further, royal jelly is thought to have a role in inhibiting tumor formation. Thus, therapeutically effective amounts of one or both of royal jelly and honokiol are included in the composition of the present invention.

The composition includes at least one Wnt pathway inhibitor and at least one histone deacetylase inhibitor. Each embodiment of the composition of the present invention includes therapeutically effective amounts of each substance so that the composition can inhibit the Wnt pathway and will also inhibit histone deacetylases. In one embodiment, the composition includes substantially equal amounts of demethoxycurcumin, quercetin, royal jelly, and honokiol.

The composition may additionally include therapeutically effective amounts of one or more substances or compounds having antitumorigenic or anti-cancer properties. Such substances are not specifically adapted to inhibit a particular pathway, such as the Wnt pathway or to function as histone deacetylase inhibitors. However, such substances are known or thought to have some anti-cancer activity. Such substances include graviola guanabana, gogi, salvestrol, and Ganoderma lucidum, among other naturally occurring substances having antitumorigenic effects.

Graviola guanabana is derived from a berry grown in South America and includes various compounds such as annonaceous acetogenins, which are thought to have anti-tumorous properties and selective toxicity against various cancer cells, such as liver and breast cancer cells. Some studies have shown that Graviola extracts can be used in certain circumstances to inhibit growth of cancer cells by blocking access of cancer cells to ATP.

Gogi, or wolf berry of the Lycium barbarum variety produce a compound that helps to inhibit cancer growth and that contributes to cell death. Some studies have shown that lyceum barbarum polysaccharide stopped the growth cycle of human gastric and colon cancer cells.

Salvestrol is a natural phytochemical that produces anti-cancer metabolites. The enzyme, CYP1B1 converts salvestrols into compounds that cause cell death of cancer cells. Some studies have shown that salvestrols have antitumorigenic effects.

The lingzhi mushroom or reishi mushroom has been used as an adjunct treatment for patients undergoing chemotherapy treatments. The reishi mushroom is a species complex of the genus Ganoderma, and is closely related to the species Ganoderma lucidum.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A composition for treating cancer, consisting essentially of:
therapeutically effective amounts of at least one histone deacetylase inhibitor and at least one Wnt signaling pathway inhibitor.

2. The composition for treating cancer of claim 1, wherein said at least one histone deacetylase inhibitor includes royal jelly.

3. The composition for treating cancer of claim 1, wherein said at least one histone deacetylase inhibitor includes honokiol.

4. The composition for treating cancer of claim 1, wherein said at least one Wnt signaling pathway inhibitor includes demethoxycurcumin.

5. The composition for treating cancer of claim 1, wherein said at least one Wnt signaling pathway inhibitor includes quercitin.

6. The composition for treating cancer of claim 1, further consisting essentially of one or more substances having anti-cancer activity, wherein said one or more substances are selected from the group consisting of: graviola guanabana, salvestrols, gogi, and ganoderma lucidum.

7. A composition for treating cancer, consisting essentially of therapeutically effective amounts of demethoxycurcumin, quercitin, honokiol, and royal jelly.

8. The composition for treating cancer of claim 7, further consisting essentially of one or more substances selected from graviola guanabana, salvestrols, lyceum barbarum, and ganoderma lucidum.
